(19) United States
(12) Patent Application Publication
(10) Pub. No.: US 2024/0415389 A1
(43) Pub. Date: Dec. 19, 2024

(54) ARTIFICIAL INTELLIGENT FINGER-RING DEVICE FOR PRE-ALARMING HEART DISEASE

(71) Applicant: Kuo-Yuan CHANG, New Taipei City (TW)

(72) Inventor: Kuo-Yuan CHANG, New Taipei City (TW)

(21) Appl. No.: 18/490,098

(22) Filed: Oct. 19, 2023

(30) Foreign Application Priority Data
Jun. 15, 2023 (TW) 112122454

(51) Int. Cl. A61B 5/00

(52) U.S. Cl. CPC A61B 5/0062; A61B 5/0015; A61B 5/7253; A61B 5/7405; A61B 5/746; A61B 5/6826; A61B 2560/02; A61B 2560/0443; A61B 2560/0462; A61B 2562/0233

(57) ABSTRACT

An artificial intelligent finger-ring device for pre-alarming heart disease, which may detect heart diseases and emit alerts even before heart disease symptoms happen, comprises a detection block, a communication-transmission block, a control block and a power block. The detection block includes a light source emitting green light and a light receiver receiving a response light signal. The green light is incident to a finger of a user and reflected to form the response light signal. The communication-transmission block transmits the response light signal to an external device and receives an analysis result from the external device. The analysis result which is acquired by analyzing the response light signal includes a time-domain index and a frequency-domain index applicable to monitor heart diseases. The control block receives the analysis result and determine whether to emit alerts. The power block supplies electric power to the detection block, the communication-transmission block and the control block.

### CROSS REFERENCES

This Application claims benefit of TW Patent Application No. 112122454 filed on Jun. 15, 2023 and titled as “Artificial Intelligent Finger Ring Apparatus of Monitoring Heart Disease”, the disclosure of which is hereby incorporated by references.

### 1. Field of the Invention

The present invention relates to a wearable device for monitoring vital signs, particularly to a finger-ring device able to pre-alarming atrial fibrillation and monitoring heart diseases.

### 2. Description of the Prior Art

Electrocardiography (EKG or ECG) is usually used to monitor the heart. Electrocardiographic device is bulky and needs sticking to the breast. Therefore, it is unfavorable for 24-hour home monitoring. A ring device can be worn on a finger to monitor the heart anytime, especially in sleep. It is acknowledged that sudden death is generally not to be pre-alarmed and may happens due to acute atrial fibrillation.

Sudden death is normally referred to the case that a person falls suddenly without external factors and dies within 24 hours. For sudden death, the duration from falling ill acutely to death is unsettled but normally presumed to one hour. According to researches, sudden death is normally induced by cardiogenic factors, such as coronaria-related cardiovascular diseases, heart failure, and cardiac arrhythmia. Cardiac arrhythmia is a generic term for abnormal heart rate, including atrial fibrillation (AF), superventricular tachycardia, atrial flutter (AF2), atrial tachycardia (AT), ventricular tachycardia (VT), and heart block. The probability of ventricular-type cardiac arrhythmia-induced sudden death is higher than that of atrial fibrillation, but the incident rate of ventricular-type cardiac arrhythmia-induced sudden death is lower than that of atrial fibrillation.

The symptoms of atrial fibrillation include heart palpitation, chest tightness, chest pain, tachypnea, dizziness, general weakness in serious state, weariness, heart failure, dyspnea, hypotension syncope, and consciousness variation. The abovementioned symptoms may be similar to the symptoms caused by other diseases. Therefore, atrial fibrillation (AF) may be neglected or misjudged in the first moment and then leads to serious results. Besides, one third of the patients who may have AF are free of any symptom. Further, atrial fibrillation takes place paroxysmally for most of the symptomatic patients and electrocardiography often fails to detect the occurrence of atrial fibrillation at the right time. Thus, the number of the patients of atrial fibrillation is seriously underestimated. If a patient who does suffer atrial fibrillation or cardiac arrhythmia is neither diagnosed nor treated, the risk of apoplexy and sudden death of the patient will significantly increase.

The conventional technologies do not use finger ring devices, AI algorithms, wireless transmission technology, IoT technologies, and cloud platform technologies to monitor and predict heart diseases.

### SUMMARY OF THE INVENTION

An artificial-intelligence (AI) finger-ring device for pre-alarming heart disease is provided, which is a wearable finger-ring device for monitoring atrial fibrillation (AF) that may induce apoplexy or acute cardiac arrhythmia that may induce sudden death. The user may wear for a long time such a finger-ring device onto especially the finger pulp of a single finger without discomfort. The user may put on or take off the finger ring-type device by himself without the help professional personnel. The finger-ring device is easy and convenient for a user to measure and analyze vital signals within one or more intervals and transmit the measurement results to an external device. The external device analyzes the measurement results and presents the analysis results on a mobile phone, an iPad or a computer, and so on. The device of the present invention may warn in advance the user or others of the risk of atrial fibrillation or sudden death, whereby the user or others may arrange or perform professional diagnosis and treatment as early as possible.

An artificial intelligent finger-ring device for pre-alarming heart disease is provided, which processes and analyzes the measured response light signals to acquire the heart disease-related time-domain index and frequency-domain index. A comprehensive result of the time-domain index and frequency-domain index relevant to monitoring heart diseases may be used to monitor the heart diseases and make pre-alarm before heart diseases occur.

The present invention provides an artificial intelligent finger-ring device for pre-alarming heart disease, which includes a detection block including a light receiver and a light source emitting a green light, wherein a finger of a user reflects the green light to generate a response light signal, and the light receiver receives the response light signal; a communication-transmission block transmitting the response light signal to an external device and receiving an analysis result from the external device, wherein the external device analyzes the response light signal to generate the analysis result; the analysis result includes a time-domain index and a frequency-domain index relevant to monitoring heart diseases; a control block electrically connected with the detection block and the communication-transmission block, receiving the analysis result, and determine according to the time-domain index and the frequency-domain to generate a warning or not; and a power block suppling electric power to the detection block, the communication-transmission block, and the control block.

The present invention also provides an artificial intelligent finger-ring device for pre-alarming heart disease, which includes a detection block including a light receiver and a light source emitting a green light, wherein a finger of a user reflects the green light to generate a response light signal, and the light receiver receives the response light signal; a controlling-processing block electrically connected with the detection block, and receiving and analyzing the response light signal to acquire an analysis result, wherein the analysis result includes a time-domain index and a frequency-domain index relevant to monitoring heart diseases, and according to the time-domain index and a frequency-domain index, the controlling-processing block determine to generate a warning or not; a communication-transmission block, transmitting at least one of the analysis result and a judgment result of the controlling-processing block to an external device; and a power block suppling electric power to the detection block, the controlling-processing block, and the communication-transmission block.

The artificial intelligent finger-ring device for pre-alarming heart disease according to claim 8 further comprising a sensing block which is electrically connected with the controlling-processing block, sensing a state of the finger of the user, and transmitting a sensing result to the controlling-processing block, wherein according to the sensing result, the controlling-processing block controls the detection block to perform detection in real time or controls the communication-transmission block to transmit the light response signal and/or the analysis result to the external device.

### BRIEF DESCRIPTION OF THE DRAWINGS

FIG. 1 is a perspective view schematically showing an artificial intelligent finger-ring device for pre-alarming heart disease according to a first embodiment of the present invention.

FIG. 2 is a perspective view schematically showing that the artificial intelligent finger-ring device for pre-alarming heart disease of the first embodiment of the present invention is worn on a finger of a user.

FIG. 3 is a block diagram schematically showing the system of the artificial intelligent finger-ring device for pre-alarming heart disease according to the first embodiment of the present invention.

FIG. 4 is a block diagram schematically showing the system of a artificial intelligent finger-ring device for pre-alarming heart disease according to a second embodiment of the present invention.

FIG. 5 is a block diagram schematically showing the system of a artificial intelligent finger-ring device for pre-alarming heart disease according to a third embodiment of the present invention.

FIG. 6 is a flowchart of the process that the signal of the response light is analyzed by the artificial intelligent finger-ring device for pre-alarming heart disease of the first embodiment of the present invention analyzes.

### DESCRIPTION OF THE PREFERRED EMBODIMENT

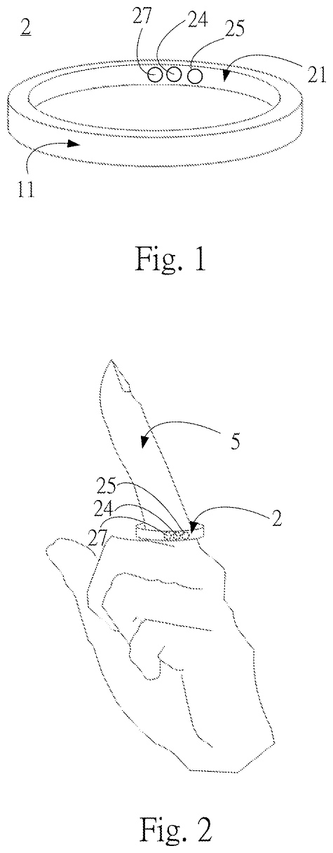

FIG. 1 is a perspective view schematically showing an artificial intelligent finger-ring device for pre-alarming heart disease according to a first embodiment of the present invention. FIG. 2 is a perspective view schematically showing that the artificial intelligent finger-ring device for pre-alarming heart disease according to the first embodiment of the present invention is worn on a finger. Refer to FIG. 1 and FIG. 2. The artificial intelligent finger-ring device for pre-alarming heart disease 2 includes a ring-shape body to be worn on a finger 5. An outer surface 11 of the body faces is outward and an inner surface 21 faces the skin of the finger 5 while a user wears the artificial intelligent finger-ring device for pre-alarming heart disease 2 on his/her finger 5. The ring-shape body of the artificial intelligent finger-ring device for pre-alarming heart disease 2 provides a space accommodating detection, control, storage or warning blocks, etc. A detection light receiver 24, a green light source 25 and a red light source 27 are exposed to the inner surface 21 to face the finger 5 especially the finger pulp. It is noted that the light receiver 24, the green light source 25 and the red light source 27 face the lateral surface of the finger 5. In other words, the light receiver 24, the green light source 25 and the red light source 27 are disposed between two fingers/or the lateral side of the finger 5, instead of one surface aligning with the palm or the back of the palm. The arrangement of the light receiver 24, the green light source 25 and the red light source 27 shown in FIG. 1 and FIG. 2 is only an exemplification. In one embodiment, the light receiver 24 is disposed in an outmost position of the three elements. In one embodiment, the green light source 25 and the red light source 27 are integrated into a hybrid light source able to emit green light and red light simultaneously or respectively emit green light and red light in sequence. In the present invention, the light source may be a combination of single-wavelength light sources, a combination of single-wavelength light sources and a multi-wavelength light source, or a combination of one or more multi-wavelength light sources. Furthermore, the light source and the light receiver may be integrated together.

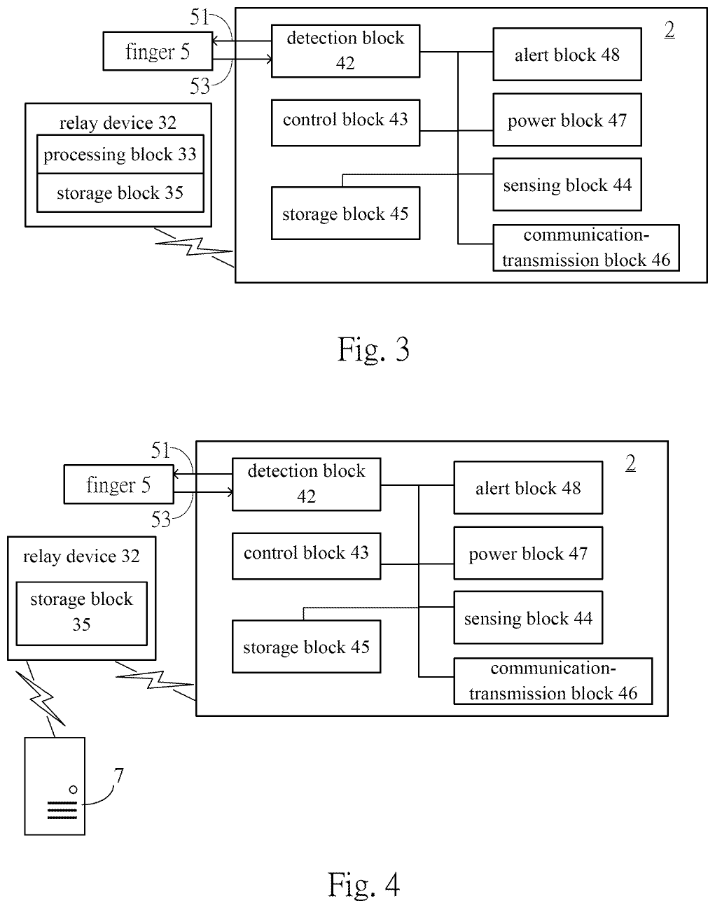

FIG. 3 is a block diagram schematically showing the system of the artificial intelligent finger-ring device for pre-alarming heart disease according to the first embodiment of the present invention. Refer to FIG. 1, FIG. 2 and FIG. 3 simultaneously. The artificial intelligent finger-ring device for pre-alarming heart disease 2 includes a detection block 42, a control block 43, a storage block 45, a power block 47, an alert block 48, a communication-transmission block 46, and a sensing block 44 accommodated in the ring-shape body. The detection block 42 emits a detection light 51 and receives the response light 53, which may include one or more light sources. For example, the detection block 42 includes a green light source 25, a red light source 27, and a light receiver 24 for receiving the response light 53 (as shown in FIG. 1), and may get electric power from the power block 47. The control block 43 is electrically connected with the detection block 42, the storage block 45, the power block 47, the alert block 48 and the sensing block 44, and communicates with each other. The control block 43 at least controls the operation frequency of the detection block 42. For example, the control block 43 controls the frequency of the detection light 51 emitted by the detection block 42 and the frequency of the response light 53 received by the detection block 42. The control block 43 stores the signal of the response light 53 into the storage block 45 and retrieves the signal of the response light 53 from the storage block 45. The control block 43 includes a processor, peripheral circuits, storage firmware, a storage unit for programs and instructions. The storage block 45, such as the memory and the peripheral circuit, is used to store the signal of the response light 53. The sensing block 44 may include an accelerometer able to sense acceleration, or an electric-magnetic sensing device/unit/module able to perform wireless sensation, or a microelectromechanical module/element, such as one or more gravitational accelerometers or gyroscopes. The sensing block 44 can sense the states of the user and/or the finger of the user. For example, the sensing block 44 may sense whether the user is moving the finger or the finger is stationary and then transmit the sensing result to the control block 43. According to the sensing result, the control block 43 controls the detection block 42 to perform detection and/or access the signal of the response light 53, whereby to perform detection in real time and/or perform communication-transmission. For example, while sensing that the finger 5 of the user is stationary, the sensing block 44 may transmit the sensing result to the control block 43, and the control block 43 immediately controls the detection block 42 to perform detection so as to obtain better signals of the response light 53.

Refer to FIG. 1, FIG. 2 and FIG. 3 again. The communication-transmission block 46 communicates with an external module/device and transmits the signal of the response light 53 to the external module/device. The external module/device analyzes the signal of the response light 53 to acquire an analysis result. The communication-transmission block 46 then returns the analysis result to the control block 43. According to the analysis result of the signal of the response light 53, the control block 43 determines whether to instruct the alert block 48 to make a warning to the user or others. The communication-transmission block 46 may include a near-distance or wireless communication module/element/circuit, such as a Bluetooth communication module, a near-field communication module, a wireless communication module, or a combination of at least two of the abovementioned modules. The power block 47 includes a recharger module and an electricity storage module, which may supply electric power to the blocks/modules/units of the artificial intelligent finger-ring device for pre-alarming heart disease 2. The control block 43 may further include an auto gain control (AGC) module, which can perform the AGC function. If it is found that the pulse signals is unqualified during processing the pulse signals, the AGC function of the AGC module can enhance the effective portions of the response light 53.

Refer to FIG. 1, FIG. 2 and FIG. 3 again. The relay device 32 at least includes a processing block 33 and a storage block 35. The relay device 32 such as a receiver, receives the signal of the response light 53 from the communication-transmission block 46 in a wireless communication method. The relay device 32 may include the storage block 35 for storing the signal of the response light 53 and the processing block 33 for processing and analyzing the signal of the response light 53 to acquire an analysis result. Then, the relay device 32 transmits the analysis result to the communication-transmission block 46. It is easily understood: the relay device 32 may further include a display block (not shown in the drawing) for presenting the analysis result. FIG. 4 is a block diagram schematically showing the system of an artificial intelligent finger-ring device for pre-alarming heart disease according to a second embodiment of the present invention. The second embodiment is different from the first embodiment in that the relay device 32 of the second embodiment is in communication connection with a remote server 7 through a network. In the second embodiment, after receiving the signal of the response light 53 from the artificial intelligent finger-ring device for pre-alarming heart disease 2, the relay device 32 may wired or wirelessly upload the signal of the response light 53 to the remote server 7 for processing and analysis. The server 7 transmits the analysis result to the relay device 32, and the relay device 32 transmits the analysis result to the artificial intelligent finger-ring device for pre-alarming heart disease 2. Then, the artificial intelligent finger-ring device for pre-alarming heart disease 2 determines to make an alert or not.

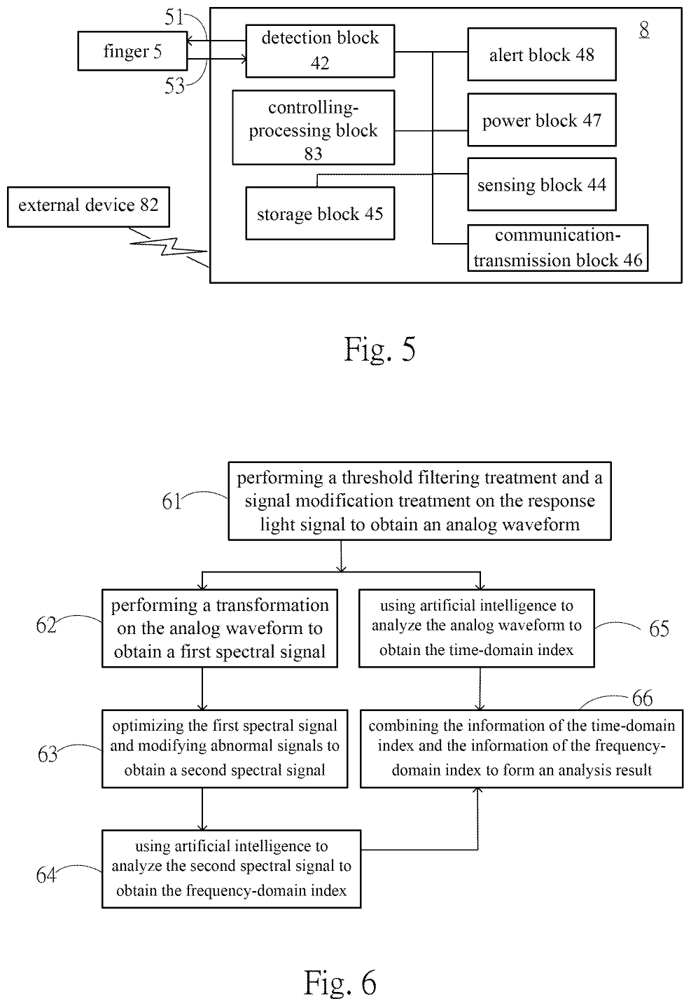

FIG. 5 is a block diagram schematically showing the system of a artificial intelligent finger-ring device for pre-alarming heart disease according to a third embodiment of the present invention. Refer to FIGS. 1-3 and FIG. 5. Compared with the artificial intelligent finger-ring device for pre-alarming heart disease 2, the controlling-processing block 83 of the artificial intelligent finger-ring device for pre-alarming heart disease 8 may process and analyze the signal of the response light 53 to acquire an analysis result and a judgment result for determining whether to activate the alert block 48 to warn the user or others. Further, the communication-transmission block 46 may transmit the analysis result and/or judgment result of the controlling-processing block 83 to an external device 82. The external device 82, such as a mobile phone, may use an application program to present the analysis result or even make an alert.

FIG. 6 is a flowchart of the process that the artificial intelligent finger-ring device for pre-alarming heart disease analyzes the signal of the response light. Refer to FIGS. 1-6. The green light source 25 of the artificial intelligent finger-ring device for pre-alarming heart disease 2 or 8 emits a green light, such as a detection light 51 with wavelengths ranging from 490 to 570 nm and frequencies ranging from 526 to 606 THz. The detection light 51 is incident to the finger 5 of the user and hits the vessels on the lateral side of the finger to generate the response light 53. The response light 53 is received by the receiver 24 to form the signal of the response light 53. It is easily understood: the detection light 51 may include a red light emitted by the red light source 27. The red light is incident to the finger 5 of the user to generate the response light 53 including the response light of the red light which may be used to measure blood oxygen. It is easily understood: the detection light 51 may include light with different wavelengths to generate corresponding signals in response to different vital signs. In one embodiment, the control block 43 or the controlling-processing block 83 may include built-in detection control instructions, which control the detection block 42 to perform detection activities at a preset frequency, such as a detection activity per 30 seconds or a detection activity per 3 minutes. In one embodiment, the control block 43 or the controlling-processing block 83 may modify the control instructions to the detection block 42 according to the sensing result of the sensing block 44. For example, the control block 43 or the controlling-processing block 83 may control the detection block 42 to perform detection immediately according to the sensing result that the finger or the user is stationary. In one embodiment, the control block 43 or the controlling-processing block 83 may modify the control activities to the detection block 42 according to the communication result of the communication-transmission block 46. For example, while the communication-transmission block 46 detects the Bluetooth matching of the relay device 32/external device 82 or the approaching of the relay device 32/external device 82, the control block 43 or the controlling-processing block 83 controls the detection block 42 to perform detection in real time according to the matching or approaching. In one embodiment, the control block 43 or the controlling-processing block 83 may receive a control instruction from the matched/approaching relay device 32/external device 82 to vary the detection frequency of the detection block 42. For example, suppose that the external device 82 is a smart phone where a control application program is installed; the user may use the external device 82 to vary the detection frequency of the detection block 42 or make the detection block 42 perform detection immediately. For example, the detection frequency is changed from one detection per 3 minutes to one detection per 2 minutes, or the detection is performed immediately.

Refer to FIGS. 1-6 again. A threshold filtering treatment and a signal modification treatment are performed on the response light signal to acquire an analog waveform (Step S61). For example, a low-and high-pass filtering (threshold filtering treatment) passes the response light signal; next, a weight analysis is undertaken to raise the signal-to-noise ratio and decrease the abnormal spikes, whereby to acquire time-dependent analog waveform (time-domain chart). Next, artificial intelligence is used to analyze the time-dependent analog waveform (time-domain chart) to acquire the time-domain index that may be used to monitor heart diseases (Step S65). The time-domain index correlates with heart diseases and involves one or more heart-related time-domain values. On the other hand, a transformation, such as a Fast Fourier Transform and/or a Wavelet Transform, is performed on the analog waveform to obtain a spectrogram of a first spectral signal (Step S62). Next, the first spectral signal is optimized to obtain a second spectral signal (Step S63). In one embodiment, according to the number of the points in the spectrogram, the energy intensities are normalized, and the abnormal intensities of the very low frequencies are modified, whereby to obtain the second spectral signal (the corrected spectrogram). However, the present invention is not limited by the abovementioned embodiment. Next, artificial intelligence is used to analyze the second spectral signal (the corrected spectrogram) to obtain the frequency-domain index that may be used to monitor heart diseases (Step S64). The frequency-domain index also correlates with heart diseases and involves one or more heart-related frequency-domain values. Then, the information of the time-domain index together with the information of the frequency-domain index is comprehensively considered to form an analysis result (Step S66). In Step S66, the analysis result includes one or more heart-related time-domain values and one or more heart-related frequency-domain values, which mainly tell acute cardiac arrhythmia, especially atrial fibrillation. Once the control block or the controlling-processing block judges the analysis result and determines that it is acute atrial fibrillation, the control block or the controlling-processing block instructs the alert block to remind the user or others. In one embodiment, the analysis result includes a first heart-related time-domain value, a second heart-related time-domain value, a first heart-related frequency-domain value, and a second heart-related frequency-domain value; while the control block or the controlling-processing block determines that the first heart-related time-domain value is greater than a preset value and that the sum of all the heart-related time-domain values and all the heart-related frequency-domain values is greater than a second preset value, it indicates that atrial fibrillation may occur and that the user may be at high risk of sudden death. According to the judgment result, the control block or the controlling-processing block may control the alert block to make an alert. In some embodiments, the alert block makes the artificial intelligent finger-ring device for pre-alarming heart disease vibrate, make sounds (buzz), or generate visualized signals (in form of text or lights) to draw attention. For example, while the control block determines that some of these indexes exceed preset values and that the user is at risk of sudden death, the control block sends an instruction to control the alert block to make buzzes to remind the user or the persons around the user.

Refer to FIGS. 1-6 again. In the case of a user always and continuously wearing the artificial intelligent finger-ring device for pre-alarming heart disease on his/her finger 5 for a long time, the artificial intelligent finger-ring device for pre-alarming heart disease may periodically perform detection and store the response light signals. The stored response light signals are wirelessly transmitted to the relay device or the external device according to the preset frequency. The relay device or the external device may further transmit the information of the stored response light signals to the server according to the preset frequency. Alternatively, the external device may directly process and analyze the information of the stored response light signals and then return the analysis result. In one embodiment, the stored response light signals subject to an instant instruction are transmitted to the relay device or the external device in real time, and processed and analyzed by the relay device or the external device immediately. It is easily understood: while the relay device is a smart phone (external device), the artificial intelligent finger-ring device for pre-alarming heart disease may send out the response light signals inside the storage block in the Bluetooth method or another wireless method when sensing the approaching of the smart phone in a near-field sensing method or another non-contact sensing method. In the case of receiving the response light signals, the smart phone further uploads the response light signals to a cloud server, or at first directly processes and analyzes the response light signals and then uploads the analysis result to the cloud server. In one embodiment, the sensing block 44 of the artificial intelligent finger-ring device for pre-alarming heart disease may sense the variation of the posture of the user. For example, the user stops running and then sits still. The control block 43 verifies the sensing result of the posture variation and determines whether to send out the response light signals or process-analyze the response light signals. The relay device like an independent receiver may be displayed on a corresponding application program installed in another's smart phone with which another person may also receive the analysis result of the response light signals and help the user seek medical attention or others.

Refer to FIGS. 1-6 again. According to the above description, the flowchart of the process of analyzing the light response signal may be executed by the artificial intelligent finger-ring device for pre-alarming heart disease, the relay device/external device, and the server jointly. For example, the control block 43 of the artificial intelligent finger-ring device for pre-alarming heart disease 2 executes the threshold filtering treatment and the signal modification treatment; the relay device/external device and the server do other steps. Alternatively, Steps S61-S66 are all undertaken by the artificial intelligent finger-ring device for pre-alarming heart disease 8.

Accordingly, it is beneficial for a user to easily and continuously wear the artificial intelligent finger-ring device for pre-alarming heart disease of the present invention and to acquire the data for analyzing the status of the heart and learning the current situation of the user. Compared to conventional devices which data acquisition and detection are performed by attaching patches onto the specific position of his/her body with the assistance of professional personnel, the artificial intelligent finger-ring device for pre-alarming heart disease of the present invention is convenient for a user to wear it for a long time without discomfort and take off easily. In the present invention, the artificial intelligent finger-ring device for pre-alarming heart disease may easily perform long-time sampling and in-time analysis based on received response signals, which provides user with comfortable wearing experience and early warning on sudden death anytime and anywhere. Thus, the user may take any action in advance before the risk of sudden death raises. Besides, the artificial intelligent finger-ring device for pre-alarming heart disease of the present invention adopts green light as the detection light. The varying extent of the response light signal of green light is greater than that of red light, favoring the succeeding signal processing and raising the accuracy of signal analysis. In conclusion, the analysis result of the artificial intelligent finger-ring device for pre-alarming heart disease of the present invention may represent the probability that the user suffers heart diseases. Even before the symptoms of heart diseases appear, the artificial intelligent finger-ring device for pre-alarming heart disease of the present invention can monitor and pre-alarm the risk of heart diseases.

The embodiments described above are to demonstrate the technical contents and characteristics of the present invention to enable the persons skilled in the art to understand, make, and use the present invention. However, these embodiments are only to exemplify the present invention but not to limit the scope of the present invention. Any equivalent modification or variation according to the spirit of the present invention is to be also included by the scope of the present invention.

What is claimed is:

1. An artificial intelligent finger-ring device for pre-alarming heart disease, comprising:
a detection block including a light receiver and a light source emitting a green light, wherein a finger of a user reflects the green light to generate a response light signal, and the light receiver receives the response light signal;
a communication-transmission block transmitting the response light signal to an external device and receiving an analysis result from the external device, wherein the external device analyzes the response light signal to generate the analysis result; the analysis result includes a time-domain index and a frequency-domain index relevant to monitoring heart diseases;
a control block electrically connected with the detection block and the communication-transmission block, receiving the analysis result, and determine according to the time-domain index and the frequency-domain to generate a warning or not; and
a power block suppling electric power to the detection block, the communication-transmission block, and the control block.

2. The artificial intelligent finger-ring device for pre-alarming heart disease according to claim 1 further comprising a sensing block which is electrically connected with the control block, sensing a state of the finger of the user, and transmitting a sensing result to the control block, wherein according to the sensing result, the control block controls the detection block to perform detection in real time or further comprises controlling the communication-transmission block to transmit the light response signal to the external device.

3. The artificial intelligent finger-ring device for pre-alarming heart disease according to claim 1 further comprising a storage block which is electrically connected with the control block and storing the response light signal.

4. The artificial intelligent finger-ring device for pre-alarming heart disease according to claim 1 further comprising an alert block which is electrically connected with the control block and controlled by the control block to make a visible or audible signal as the warning.

5. The artificial intelligent finger-ring device for pre-alarming heart disease according to claim 1, wherein the light source of the detection block further comprises emitting a red light or a light with different wavelengths, and the finger of the user reflects the red light or the light with different wavelengths to generate the response light signal.

6. The artificial intelligent finger-ring device for pre-alarming heart disease according to claim 1, wherein the control block is electrically connected with the detection block and controls the detection block to emit light and receive response light signal in real time or according to a frequency.

7. The artificial intelligent finger-ring device for pre-alarming heart disease according to claim 1, wherein the communication-transmission block includes a Bluetooth communication module, a near-field communication module, a wireless communication module, or a combination of at least two thereof.

8. An artificial intelligent finger-ring device for pre-alarming heart disease, comprising:
a detection block including a light receiver and a light source emitting a green light, wherein a finger of a user reflects the green light to generate a response light signal, and the light receiver receives the response light signal;
a controlling-processing block electrically connected with the detection block, and receiving and analyzing the response light signal to acquire an analysis result, wherein the analysis result includes a time-domain index and a frequency-domain index relevant to monitoring heart diseases, and according to the time-domain index and a frequency-domain index, the controlling-processing block determine to generate a warning or not;
a communication-transmission block, transmitting at least one of the analysis result and a judgment result of the controlling-processing block to an external device; and
a power block suppling electric power to the detection block, the controlling-processing block, and the communication-transmission block.

9. The artificial intelligent finger-ring device for pre-alarming heart disease according to claim 8 further comprising a sensing block which is electrically connected with the controlling-processing block, sensing a state of the finger of the user, and transmitting a sensing result to the controlling-processing block, wherein according to the sensing result, the controlling-processing block controls the detection block to perform detection in real time or controls the communication-transmission block to transmit the light response signal and/or the analysis result to the external device.

10. The artificial intelligent finger-ring device for pre-alarming heart disease according to claim 8, further comprising a storage block which is electrically connected with the controlling-processing block and stores the response light signal and/or the analysis result.

11. The artificial intelligent finger-ring device for pre-alarming heart disease according to claim 8, further comprising an alert block which is electrically connected with the controlling-processing block and controlled by the controlling-processing block to make a visible or audible signal as the warning.

12. The artificial intelligent finger-ring device for pre-alarming heart disease according to claim 8, wherein the light source of the detection block also emits a red light or a light with different wavelengths, and the finger of the user reflects the red light or the light with different wavelengths to generate the response light signal.

13. The artificial intelligent finger-ring device for pre-alarming heart disease according to claim 8, wherein the controlling-processing block is electrically connected with the detection block and controls the detection block to emit light and receive reflected light signals in real time or according to a frequency.

14. The artificial intelligent finger-ring device for pre-alarming heart disease according to claim 8, wherein the controlling-processing block processes an analog waveform signal of the response light signal to acquire the time-domain index, and transforms and processes the analog waveform signal to acquire the frequency-domain index.

15. The artificial intelligent finger-ring device for pre-alarming heart disease according to claim 8, wherein the communication-transmission block includes a Bluetooth communication module, a near-field communication module, a wireless communication module, or a combination of at least two thereof.
